(19) United States
(12) Patent Application Publication
(10) Pub. No.: US 2022/0047840 A1
(43) Pub. Date: Feb. 17, 2022

(54) Combined Warmer and Seat Fumigator Using Bioceramics

(71) Applicant: Kyu Sik LEE, Gyeonggi-do (KR)

(72) Inventor: Kyu Sik LEE, Gyeonggi-do (KR)

(21) Appl. No.: 17/309,618

(22) PCT Filed: Dec. 10, 2019
(86) PCT No.: PCT/KR2019/017363

§ 371 Date: Jun. 10, 2021

(30) Foreign Application Priority Data
Dec. 11, 2018 (KR) 10-2018-0159427

(51) Int. Cl. A61M 21/02; C04B 35/14; C04B 35/626; C04B 35/64; C04B 38/06; A61F 7/08

(52) U.S. Cl. CPC A61M 21/02; C04B 35/14; C04B 35/62675; C04B 35/64; C04B 38/069; A61M 2021/0066; C04B 2235/3418; C04B 2235/349; C04B 2235/3463; C04B 2235/656; A61F 7/08

(57) ABSTRACT

The present invention relates to a combined warmer and seat fumigator using bioceramics and, more specifically, to a combined warmer and seat fumigator using bioceramics, the combined warmer and seat fumigator being capable of carrying out both thermotherapy and fumigation therapy, having excellent heat insulation, ultralight weight, nonflammability, low gas toxicity, thermal conductivity and deodorizability, and having immune function improvement and inflammation alleviation efficacy through far infrared ray emissivity, radiant energy, anion generation and antibacterial ability.

### TECHNICAL FIELD

The present invention relates to a combined warmer and seat fumigator using bioceramic and, more specifically, to a combined warmer and seat fumigator using bioceramic, which is capable of carrying out both thermotherapy and fumigation therapy, has excellent heat insulation, ultralight weight, nonflammability, low gas toxicity, thermal conductivity and deodorizing ability, and has immune function improvement and inflammation alleviation efficacy through far infrared ray emissivity, radiant energy, anion generation and antibacterial ability.

### BACKGROUND ART

A dome-shaped electric heating device having a heat radiating device attached to the dome-shaped inner surface (Korean Patent No. 10-1354987) and a combined fumigator and thermotherapy device (Korean Patent publication No. 10-2014-0024612) were disclosed.

However, said electric heating device or said thermotherapy device has no affinity for human bodies since having a wall body made of metal or minerals, and has a narrow heat radiation area since an electric heater is a circular heater.

In order to solve such problems, Korean Patent No. 10-1662061 discloses a combined sitz bath and heating sauna apparatus including: a sauna body having a semicircular shape by assembling a pair of sauna bodies A and B of a quarter-circular shape and having a heater; and a flat-type sitting mat 4 having a recess 4a having the same shape as the folded sauna bodies A, an external recess wall 4b formed on the inner surface of the recess 4a and fit to the outer circumferential surface of a front support plate 2 of the bodies A and B, and a hole 4c formed at the central portion thereof. Blind plates 2c and 2d to which wheels 2c facing the ground are respectively attached to both end portions of front and rear support plates 2 and 2a of the bodies A and B so as to face each other. Inner walls of the blind plates are connected via woven hinges 3 so that the sauna bodies A and B can be folded and unfolded. The sauna apparatus has a wall body made of redwood.

Meanwhile, the scientific circles since the 18th century regarded light as waves. In December of 1900, Max Planck who is a physicist in Germany revealed that light is grains or particles of energy and quanta of light are transmitted sparsely. Starting in 20th century, physicists found out that materials are broken down into molecules, molecules are broken down into atoms, and atoms are broken down into atomic nucleuses and electrons. During the process of revealing the nature of electrons, the physicists proved that the electrons have duality of particles and waves. That is, all existing materials have the dual structure of particles and waves. It has been known that quantum is the smallest unit of energy which is no more indivisible. When super-quantum fields are overlapped, quanta are changed into waves, when waves are overlapped, they are changed into energy, when energies are overlapped, they are changed into elementary particles, when elementary particles are overlapped, they are changed into atoms, when atoms are overlapped, they are changed into molecules, and when molecules are overlapped, they form a material.

That is, all materials (humans, animals, plants, and so on) form a material through the steps of waves, and energies by overlapping of super-quantum fields. Therefore, in an area where there are quantum energies formed by overlapping of super-quantum fields, living things including humans are activated. On the other hand, in an area where there are no quantum energies and super-quantum fields are not overlapped, humans and all living things are not activated but die of illness.

We who are living in dimensional reality recognize all things through senses and think that those are all. However, in the ultrafine world beyond the elementary particles (atom nucleuses and electrons) which are the smallest units of all materials, the world is in the quantum energy field which is unseen.

All materials in the ultrafine world are not solid but are formed by activities of electrons which ceaselessly turn around the atom nucleus.

An electromagnetic field is formed around the atom nucleus by the activities of electrons turning around the atom nucleus which is an elementary particle, and then, fine quantum energy having intrinsic information of the material is emitted. Such quantum energy fields have intrinsic information on the nature world, the material world or the mental world and exchange information with each other through waves. Albert Einstein said if materials in the world are split over and over, they are finally connected with one another as a vibrating energy field.

The bioceramic which emits far-infrared rays serves to transmit thermal effect through generation of quantum wave energy and to provide pain relief, immunity improvement, blood circulation improvement by warming a cold body.

### PATENT LITERATURE

### Patent Documents

Patent Document 1: Korean Patent No. 10-1354987

Patent Document 2: Korean Patent Publication No. 10-2014-0024612

Patent Document 3: Korean Patent No. 10-1662061

### DISCLOSURE

### Technical Problem

Accordingly, the present invention has been made in an effort to solve the above-mentioned problems occurring in the prior arts, and it is an object of the present invention to provide a combined warmer and seat fumigator using bioceramic, which is capable of carrying out both thermotherapy and fumigation therapy, has excellent heat insulation, ultralight weight, nonflammability, low gas toxicity, thermal conductivity and deodorizing ability, and has immune function improvement and inflammation alleviation efficacy through far infrared ray emissivity, radiant energy, anion generation and antibacterial ability.

The technical problem to be solved by the present invention is not limited to the technical problem as mentioned above, and another technical problem, which is not mentioned, could be clearly understood by those having ordinary skill in the art to which the present invention pertains based on the description below.

### Technical Solution

To achieve the above objects, the present invention provides a combined warmer and seat fumigator using bioceramic including: a main body including a first partitioned body of an arc shape of which a central angle is 90 degrees and a second partitioned body of an arc shape of which one end is coupled to the first partitioned body by a hinge and of which a central angle is 90 degrees, and a structure capable of being folded or unfolded; thermal modules respectively mounted on the inner circumferential surfaces of the first partitioned body and the second partitioned body to emit heat; and bioceramic mounted on the thermal modules or on the inner circumferential surface of the main body, wherein when the main body is unfolded to become a semicircle, a user can lay in the main body to get thermotherapy, and wherein when the main body is folded and a sitting mat is mounted on the upper surface of the main body, the user can sit on the sitting mat to get thermotherapy.

Moreover, the combined warmer and seat fumigator further includes fumigation modules respectively mounted on the inner circumferential surfaces of the first partitioned body and the second partitioned body.

Furthermore, each of the fumigation modules comprises: a heating plate mounted in the first partitioned body and the second partitioned body, a container getting in contact with the heating plate to accommodate herb medicines therein and having a discharge hole for discharging fumigation of the herb medicines into an internal space of the folded main body, and a cover for opening and closing an opening part of the container.

Additionally, the container or the cover is made of bioceramic.

In addition, the bioceramic is manufactured through the steps of: (S1) crushing perlite and heating it at 850° C. to 950° C. in order to make foamed silica stone; (S2) mixing and molding 20 to 40 parts by weight of inorganic binder based on 100 parts by weight of foamed silica stone; and (S3) sintering the molded body through the step (S2) at 900° C. to 1,200° C.

Moreover, the inorganic binder of the step (S2) consists of 100 parts by weight of clay, 30 to 50 parts by weight of hollow body, 10 to 30 parts by weight of attapulgite, 5 to 15 parts by weight of foaming agent, and 50 to 70 parts by weight of water, and in the step (S3), the molded body is sintered and foamed.

Furthermore, the mixture of the step (S2) further includes 10 to 30 parts by weight of inorganic filler, which is at least one selected from diatomite, bentonite, zeolite, and alumina based on 100 parts by weight of the foamed silica stone.

Additionally, the hollow body is hollow silica powder having silica fume coated on the surface thereof, and wherein the foaming agent is silicon carbide or calcium carbonate.

### Advantageous Effects

The combined warmer and seat fumigator using bioceramic, which is to solve the above-mentioned problems can carry out both thermotherapy and fumigation therapy, has excellent heat insulation, ultralight weight, nonflammability, low gas toxicity, thermal conductivity and deodorizing ability, and has immune function improvement and inflammation alleviation efficacy through far infrared ray emissivity, radiant energy, anion generation and antibacterial ability.

The effects of the present invention are not limited to the above-mentioned effects and further effects not described above will be clearly understood by those skilled in the art.

### DESCRIPTION OF DRAWINGS

FIG. 1 is a perspective view showing a state where a combined warmer and seat fumigator using bioceramic according to the present invention is spread.

FIG. 2 is a perspective view showing a state where the combined warmer and seat fumigator using bioceramic according to the present invention is folded.

FIG. 3 is a bottom view of the combined warmer and seat fumigator using bioceramic according to the present invention.

FIG. 4 is a sectional view of FIG. 3.

### MODE FOR INVENTION

Hereinafter, reference will be now made in detail to the preferred embodiments of the present invention with reference to the attached drawings.

In the description of the present invention, when it is judged that detailed descriptions of known functions or structures related with the present invention may make the essential points vague, the detailed descriptions of the known functions or structures will be omitted. Further, wordings to be described later are defined in consideration of the functions of the present invention, and may differ depending on the intentions of a user or an operator or custom. Accordingly, such wordings should be defined on the basis of the contents of the overall specification.

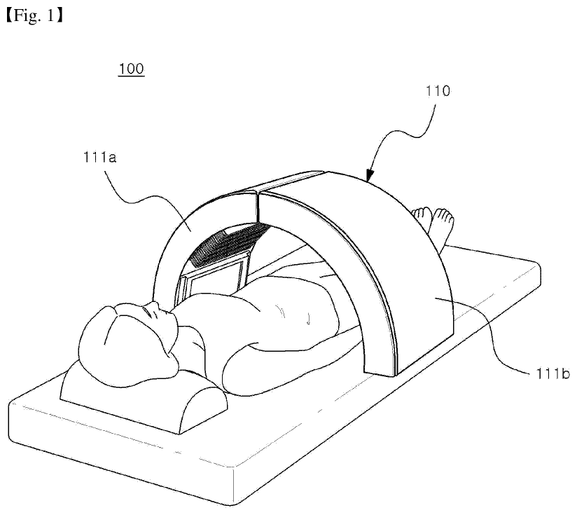

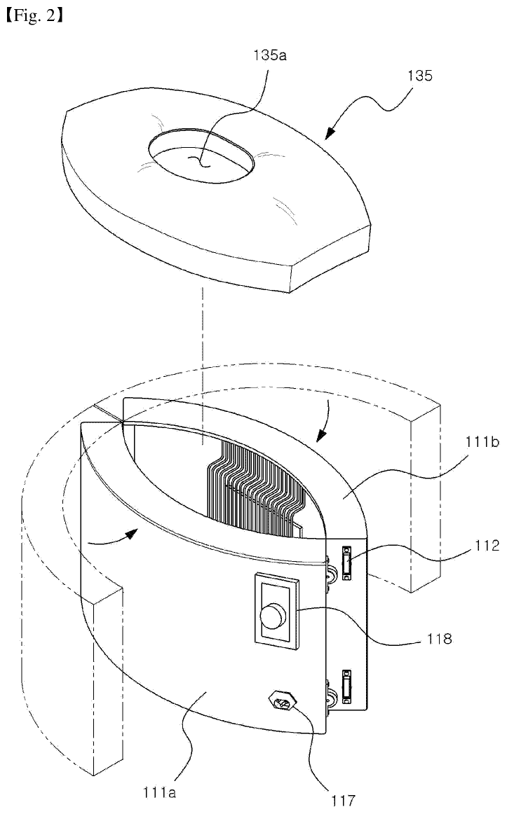

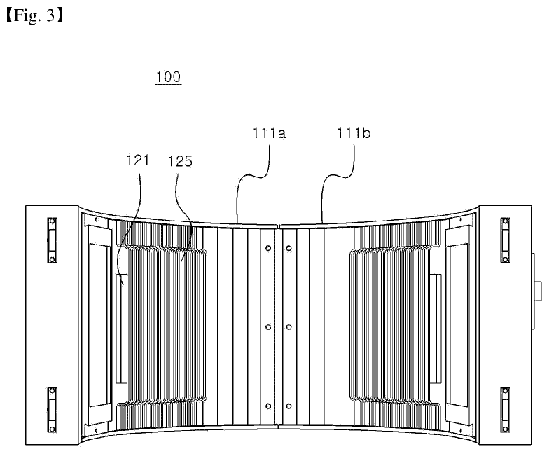

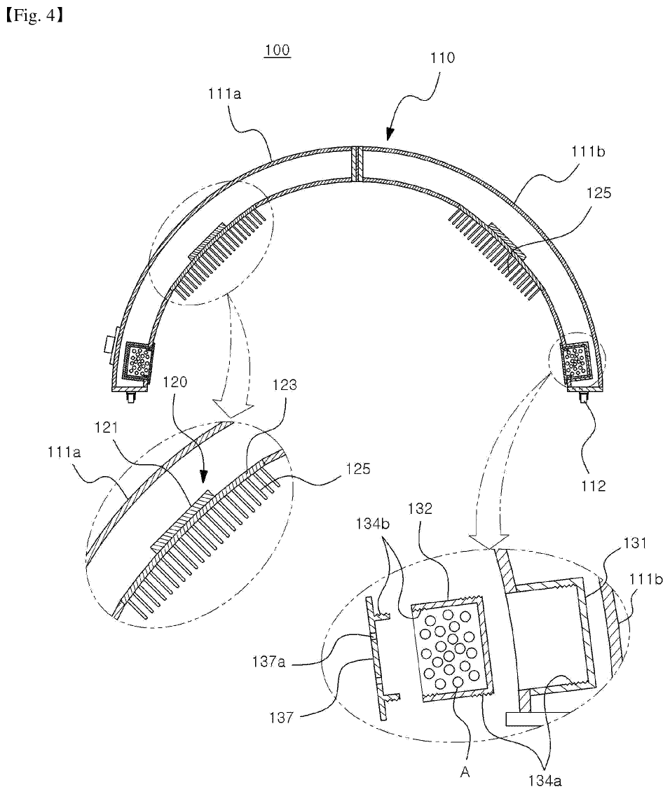

FIG. 1 is a perspective view showing a state where a combined warmer and seat fumigator using bioceramic according to the present invention is spread, FIG. 2 is a perspective view showing a state where the combined warmer and seat fumigator using bioceramic according to the present invention is folded, FIG. 3 is a bottom view of the combined warmer and seat fumigator using bioceramic according to the present invention, and FIG. 4 is a sectional view of FIG. 3.

Referring to FIGS. 1 and 2, the combined warmer and seat fumigator 100 using bioceramic according to the present invention largely includes a main body 110 and a thermal module 120.

The main body 110 includes a first partitioned body 111a of an arc shape of which a central angle is 90 degrees, and a second partitioned body 111b of an arc shape of which one end is coupled to the first partitioned body 111a by a hinge 111c and of which a central angle is 90 degrees. So, the main body 110 has a structure capable of being folded or unfolded.

When the main body 110 is unfolded to become a semicircle, it has a dome-shaped warmer structure in which a user lays in the main body 110 to get thermotherapy.

In case that the main body 110 is unfolded to become a semicircle, the combined warmer and seat fumigator 100 can be easily moved to a desired position using wheels 112 mounted on the bottoms of the first partitioned body 111a and the second partitioned body 111b.

A socket 117 for supplying power and a controller 118 for adjusting temperature of the thermal module or a fumigation module, which will be described later, turning on and off the thermal module or the fumigation module, and selecting the modules are mounted on the outer circumferential surface of any one among the first partitioned body 111a and the second partitioned body 111b.

Moreover, the first partitioned body 111a and the second partitioned body 111b may be made of wood which generates phytoncide like cypress trees. Alternatively, the first partitioned body 111a and the second partitioned body 111b may be generally made of wood, but a part of the inner circumferential surface may be made with plate-shaped or bar-shaped bioceramic.

Bioceramic can provide excellent heat insulation, ultralight weight, nonflammability, low gas toxicity, thermal conductivity and deodorizing ability, and also provide immune function improvement and inflammation alleviation efficacy through far infrared ray emissivity, radiant energy, anion generation and antibacterial ability. How to make such bioceramic will be described later.

The thermal module 120 supplies heat into the main body 110 to provide thermotherapy or dry sauna function. The thermal module 120 includes a heating plate 121, a plate-shaped bioceramic 115 arranged on one side of the heating plate 121 to emit heat into the main body 110, and a safety net 125. Alternately, the thermal module may include a far-infrared lamp instead of the heating plate, and a reflecting plate arranged at the back of the far-infrared lamp, and in this instance, the reflecting plate is made of bioceramic.

A pair of the thermal modules 120 are respectively mounted in the first partitioned body 111a and the second partitioned body 111b.

Meanwhile, as shown in FIG. 2, when the first partitioned body 111a and the second partitioned body 111b are folded to come into contact with each other and a sitting mat 135 is mounted on the upper surface of the folded body, the main body 110 is changed into a seat fumigator which allows the user to sit on the sitting mat to get thermotherapy and fumigation therapy.

The sitting mat 135 has a hole 135a formed at the center thereof so that heat or fumigation is supplied.

Referring to FIGS. 3 and 4, the combined warmer and seat fumigator using bioceramic according to the present invention includes a fumigation module 130 mounted on the inner circumferential surfaces of the first partitioned body 111a and the second partitioned body 111b in order to provide fumigation therapy.

The fumigation module 130 includes heating plates 131 respectively mounted in the first partitioned body 111a and the second partitioned body 111b, a container 132 getting in contact with the heating plate 131 and accommodating herb medicines (A) therein, and a cover having a discharge hole 137a and opening and closing an opening part of the container 132.

Heat of the heating plate 121 of the fumigation module 130 heats the herb medicines in the container 132 to generate fumigation, and the generated fumigation is transferred to the user through the discharge hole 132a and the hole 135a.

A plurality of the fumigation modules 130 may be mounted, and in this instance, different kinds of herb medicines are put in the containers of the fumigation modules. So, the user can work only the fumigation module in which herb medicine needed by the user is contained. Moreover, if a single usage of herb medicine is put in the container of the fumigation module, it can show sufficient effect while reducing consumption of herb medicine. For example, even though twice the single usage of herb medicine is put in the container, fumigation generation time is not much different from the single usage of herb medicine, and it causes waste of medicines since excessive fumigation is generated during the same period of time. Therefore, the partitioned fumigation modules can solve such problem.

Meanwhile, the heating plate 131 has a cylindrical structure such that the container 132 is inserted into the heating plate 131 by spiral coupling 134a, and the cover 137 is also spirally coupled with the container 132.

Hereinafter, a method for manufacturing bioceramic according to a preferred embodiment of the present invention will be described in detail.

The method for manufacturing bioceramic according to the present invention includes the steps of: (S1) crushing perlite and heating it at 850° C. to 950° C. in order to make foamed silica stone; (S2) mixing and molding 20 to 40 parts by weight of inorganic binder based on 100 parts by weight of foamed silica stone; and (S3) sintering the molded body through the step (S2) at 900° C. to 1,200° C.

In the step (S1), perlite which is one of volcanic rocks is crushed, is preheated at 80° C. to 90° C. for 20 to 30 minutes, is quickly heated at 850° C. to 950° C. to be foamed by water of crystallization contained therein, and then, is cooled so as to form foamed silica stone.

Korean Patent No. 10-0803513 discloses a conventional foamed ceramic manufacturing process. In Korean Patent No. 10-0803513, ceramic material is mixed with a foaming agent, and the mixture is molded, sintered and foamed so as to form a ceramic panel. However, the conventional foamed ceramic manufacturing process has a disadvantage in that cracks are formed or a molded body is agglomerated during sintering. However, the method for manufacturing bioceramic according to present invention can solve the above-mentioned disadvantage since perlite which is one of bioceramic materials is first foamed in the step (S1), and then, is mixed and sintered together with the other materials.

In the step (S2), the foamed silica stone, inorganic binder, and inorganic filler are mixed together.

The inorganic filler is at least one selected from diatomite, bentonite, zeolite, and alumina, and 10 to 30 parts by weight of inorganic filler is mixed based on 100 parts by weight of the foamed silica stone.

Preferably, the inorganic binder is 20 to 40 parts by weight based on 100 parts by weight of the foamed silica stone. If the inorganic binder is less than 20 parts by weight, it causes poor moldability and a sharp decline of binding force. If the inorganic binder is more than 40 parts by weight, it causes decline of porosity and increase in density of the product, and it makes lightweightness difficult. Therefore, it is preferable to limit the amount of the inorganic binder to the above-mentioned range.

The present invention suggests two kinds of inorganic binder.

One is inorganic binder consisting of 100 parts by weight of illite or montmorillonite and 40 to 60 parts by weight of water.

The other one is inorganic binder consisting of 100 parts by weight of clay, 30 to 50 parts by weight of hollow body, 10 to 30 parts by weight of attapulgite, 5 to 15 parts by weight of foaming agent, and 50 to 70 parts by weight of water.

There is a difference between the two kinds of inorganic binder. The former inorganic binder has pores formed just by the first foaming during the process of manufacturing foamed silica stone, but the latter inorganic binder can remarkably increase porosity since pores are formed by the first foaming and the second foaming during the process of manufacturing foamed silica stone.

Hereinafter, the present invention will be described based on the latter inorganic binder.

Attapulgite is a clay mineral, has chemical formula of (Mg,Al)₅Si₈O₂₀.4H₂O, is needle-shaped differently from montmorillonite, has a tunnel structure by a chain structure of silica tetrahedron, serves to improve foaming performance by water molecules contained therein since containing water molecules in the tunnel, and also serves to improve moldability through viscosity control during molding of the step (S2).

For example, the hollow body is hollow silica powder of which the average particle size is 50 to 300 μm.

The hollow silica powder may be obtained through the steps of mixing, melting, quickly cooling and crushing silica and calcium oxide and foaming the crushed particles, but the hollow silica powder is not limited to the above but may be manufactured by known method, such as spray pyrolysis, or the sol-gel process.

Preferably, the hollow silica powder has a complex structure that silica powder is impregnated in solution of silica fume, which is a recycled resource, and solvent, and is dried, and then, silica fume is coated on the surface. The silica fume serves to improve dispersibility by preventing fixation of hollow silica powder and to improve durability of hollow silica powder.

The foaming agent may be silicon carbide or calcium carbonate.

The foaming mechanism of silicon carbide is disclosed in Korean Patent No. 10-1067371, and the foaming mechanism of calcium carbonate is disclosed in Korean Patent Publication No. 10-2012-0077746 in detail, and so, the detailed descriptions will be omitted.

In the meantime, Korean Patent Publication No. 10-2012-0077746 also discloses silica fume. However, the silica fume disclosed in Korean Patent Publication No. 10-2012-0077746 is difficult to be dispersed due to agglomeration since the silica fume has ultra-fine particles of 1 to 3 μm, and it is preferable that the silica fume be applied to the method of being coated to hollow silica powder rather than being mixed in case of the dry molding method like the present invention.

The bioceramic may be dried after coating liquid having functional particles is sprayed on the surface of the bioceramic, or dried after being immersed in a coating liquid, so that the functional particles are adhered to the surface of the bioceramic or inserted into a part of inner pores.

The functional particles have an average grain diameter of 100 to 500 nm, and consist of oil contained therein and a silica peel containing the oil therein, and the oil consists of 10 to 30 parts by weight of paeony root extract based on 100 parts by weight of base oil.

The base oil is, preferably, lavender oil which has non-inflammatory function, asepsis, and disinfection, prevents convulsion, relieves pain, and prevents toxic reactions. The paeony root extract is obtained through the steps of drying paeony roots and performing hot water extraction, and is effective on menstrual irregularity and pain relief. Furthermore, it has been known that paeoniflorin which has been known as a main ingredient is effective on alleviation of pain, calming, anti-inflammation, fall in blood pressure, vasodilatation, and smooth muscle relaxation.

The functional particles are obtained through the steps of preparing emulsion in which 100 parts by weight of water, 1 to 10 parts by weight of oil, 0.1 to 1 parts by weight of sucrose fatty acid ester are mixed, adding and stirring 0.1 to 1 parts by weight of diethoxydimethylsilane and 0.1 to 1 parts by weight of epichlorohydrin, and adding 10 to 20 parts by weight of water glass for five to ten hours little by little to have a reaction. Based on 100 parts by weight of a final product, 1 to 5 parts by weight of Arabia gum is mixed, then, coating liquid containing the functional particles is prepared.

The paeony root extract and the lavender oil contained in to silica peels of the functional particles show treatment of menstrual irregularity and anti-inflammation since effective ingredients of the paeony root extract and the lavender oil are emitted when bioceramic are heated.

Hereinafter, the method for manufacturing bioceramic according to a further preferred embodiment of the present invention will be described in detail.

### Embodiment 1

Perlite powder crushed to 130 to 140 μm was preheated at 85° C. for 25 minutes and was quickly heated at 890 to 900° C. so that foamed silica stone was foamed and expanded twelve times in volume.

Based on 100 parts by weight of the foamed silica stone, 30 parts by weight of inorganic binder and 20 parts by weight of inorganic filler were mixed and compressively molded. Here, the inorganic binder consisted of 40 parts by weight of hollow silica powder coated with silica fume, 10 parts by weight of calcium carbonate and 50 parts by weight of water based on 100 parts by weight of attapulgite, and the inorganic filler was mixture in which diatomite and zeolite were mixed at a weight ratio of 1 to 1.

The molded body is sintered at 950° C. in an electric furnace to manufacture bioceramic.

### Experimental Example 1

Density and flexural strength of the bioceramic of the embodiment 1, and flexural strength in a wet condition were measured, and the results were written in the following Table 1.

Density was measured by KSF 2459:2002, flexural strength of the bioceramic of the embodiment 1 and flexural strength in the wet condition were measured by SKF3200:2006, and total absorption rate was measured by KSF3504:2003.

TABLE 1

| | | | Flexural | |
|---|---|---|---|---|
| | | | strength | |
| | | Flexural | in wet | Total |
| | Density | strength | condition | absorption |
| Test Item | (g/cm²) | (N/mm²) | (N/mm²) | (%) |
| Embodiment 1 | 0.32 | 2.5 | 2.3 | 89 |

Referring to Table 1, density of the bioceramic of the embodiment 1 is 0.32 g/cm², and it shows that it is ultralight and is very high at strength compared with 0.44 g/cm² of Korean Patent Publication No. 10-2012-0077746. Additionally, in an aspect of the total absorption rate, the bioceramic of the embodiment 1 retains moisture up to 89% of dead weight. Therefore, the bioceramic discharge moisture in dry weather but absorb moisture in wet weather to control humidity in a construction space.

### Experimental Example 2

A nonflammability test of the bioceramic of the embodiment 1 was made, and the result was written in the following Table 2.

Nonflammability was measured by KSF ISO 1182:2004.

TABLE 2

| | Mass decrease | Temperature rise | Flame |
|---|---|---|---|
| Test Item | rate (%) | of furnace (° C.) | duration(s) |
| Standard | Less than 30% | Less than 20° C. | Less than 10 sec |
| Embodiment 1 | 0.3 | 1.9 | 0 |

Referring to Table 2, according to Article 2(1) of the standards of flame retardant performance of building interior finishing materials disclosed in Ministry of Construction and Transportation Notice No. 2006-476, it is prescribed that noncombustible materials are less than 30% in mass decrease rate, less than 20K in temperature rise, and less than 10 seconds in flame duration. It was shown that the bioceramic of the Embodiment 1 exceeded the standards of flame retardant performance.

### Experimental Example 3

A test on gas harmful effect of the bioceramic of the embodiment 1 and a test on a guideline of interior air quality of a new apartment house (related with Article 7(2)) were made, and the results were written in the following Table 3.

The test on gas harmful effect was measured by KSF 2271:2006.

TABLE 3

| | test on gas | Total volatile | |
|---|---|---|---|
| | harmful effect | organic | |
| | (Mouse | compoound | Formaldehyde |
| | behavior | (TVOC) | (HCHO) |
| Test Item | stop time) | (mg/m², h) | (mg/m², h) |
| Standard | More than 9 | 4.0 | 1.25 |
| | minutes | | |
| Embodiment 1 | 17.93 minutes | 0.01 | Tr |

Referring to Table 3, in connection with the test on gas harmful effect, according to Article 2(2) of Construction Notice No. 2006-476, it is prescribed that mouse behavior stop time is more than 9 minutes, but the bioceramic of the embodiment 1 exceeded the time since the mouse behavior stop time of the bioceramic was at least 15 minutes and 93 seconds. It was checked that the total volatile organic composite (TVOC) and formaldehyde (HCHO) (mg/m²,h) satisfied the standards.

### Experimental Example 4

A thermal conductivity test of the bioceramic of the embodiment 1 was made, and the result was written in the following Table 4.

The thermal conductivity was measured by KSL9016:2005.

TABLE 4

| Test Item | Thermal conductivity (w/mK) |
|---|---|
| Embodiment 1 | 0.059 |

Referring to Table 4, thermal conductivity of the bioceramic of the embodiment 1 was 0.059. It was checked that the bioceramic was remarkably improved in thermal conductivity compared with 0.12 of Korean Patent Publication No. 10-2012-0077746.

### Experimental Example 5

A deodorization test of the bioceramic of the embodiment 1 was made, and the result was written in the following Table 5.

The BLANK density in the following Table 5 was measured in a state where a sample is not put, and the test was made by KFIA-1004 which is the standard of Korea Far Infrared Application Evaluation Institute, test gas was ammonia gas, and gas density was measured using a gas detector.

TABLE 5

| | Elapse | BLANK | Sample | |
|---|---|---|---|---|
| | time | Density | density | Deodorization |
| Test Item | (minute) | (ppm) | (ppm) | rate (%) |
| Deodorization | Beginning | 500 | 500 | — |
| Test | 30 | 490 | 45 | 91 |
| | 60 | 480 | 35 | 93 |
| | 90 | 460 | 25 | 95 |
| | 120 | 450 | 20 | 96 |

Referring to Table 5, it was shown that the bioceramic of the embodiment 1 showed excellent deodorization performance since the deodorization rate was up to 96%.

### Experimental Example 6

Far infrared radiation rate, radiant energy, and anion generation amount of the bioceramic of the embodiment 1 were measured, and the results were written in the following Table 6.
The tests of far infrared radiation rate and radiant energy were made by KFIA-FI-1005 which is a test method of Korea Far Infrared Application Evaluation Institute, and were measured in comparison with BLANK BODY using 40° C. FT-IR spectrometer. The anion generation amount was measured by KFIA-FI-1042 which is the standard of Korea Far Infrared Application Evaluation Institute, and was tested under conditions of indoor temperature of 23° C., humidity of 32%, and 102/cc anions in atmosphere using a charged particle measuring device. Anions emitted from a measurement object were measured, and the measured anions were indicated as the number of ions per unit volume.

TABLE 6

| | Far infrared | Radiant | |
|---|---|---|---|
| | radiation | energy | Anion |
| Test Item | rate (5~20 μm) | (w/m, μm 40° C.) | (ION/CC) |
| Embodiment 1 | 0.921 | 3.71 × 10² | 1.030 |

Referring to Table 6, the bioceramic of the embodiment 1 was excellent at far infrared radiation and generation of anions.

### Experimental Example 7

A test on anti-bacterial effect of the bioceramic of the embodiment 1 was made, and the result was written in the following Table 7.

The thermal conductivity was measured by KSL9016:2005.

TABLE 7

| | | | Density | Bacteriostatic |
|---|---|---|---|---|
| | | Initial | after 24 | decrease |
| Test Item | Sample | density | hours | rate (%) |
| Anti- | BLANK | 3.2 × 10⁶ | 1.4 × 10⁶ | — |
| bacterial | Embodiment | | 1.0 × 10⁵ | 92.1 |
| test by | 1 | | | |
| colon | | | | |
| bacillus | | | | |
| Anti- | BLANK | 3.3 × 10⁶ | 1.5 × 10⁶ | — |
| bacterial | Embodiment | | 9.5 × 10⁴ | 93.7 |
| test by | 1 | | | |
| bacillus | | | | |
| pyocyaneus | | | | |

Referring to Table 7, it was shown that the bioceramic of the embodiment 1 showed excellent anti-bacterial performance.

While the present invention has been particularly shown and described with reference to exemplary embodiments thereof, it will be understood by those of ordinary skill in the art that the present invention is not limited thereto and various substitutions, changes and modifications may be made therein without departing from the technical idea and scope of the present invention. Therefore, the spirit of the present disclosure is not determined by being limited to the above-described embodiments, but the claims described later and all of those that are equivalent to the claims and equivalent modifications thereof belong to the spirit and scope of the present disclosure.

### EXPLANATION OF REFERENCE NUMERALS

100: combined warmer and seat fumigator
110: main body
111a: first partitioned body
111b: second partitioned body
111c: hinge
112: wheel
117: socket
118: controller
120: thermal module
121: heating plate
123: bioceramic
125: safety net
130: fumigation module
131: heating plate
132: container
132a: discharge hole
134a,134b: spiral coupling
135: sitting mat
135a: hole
137: cover
Herb medicine (A)

1. A combined warmer and seat fumigator using bioceramic comprising:
a main body including a first partitioned body of an arc shape of which a central angle is 90 degrees, and a second partitioned body of an arc shape of which one end is coupled to the first partitioned body by a hinge and of which a central angle is 90 degrees, and a structure capable of being folded or unfolded;
thermal modules respectively mounted on the inner circumferential surfaces of the first partitioned body and the second partitioned body to emit heat; and
wherein bioceramic is mounted on the thermal modules or on the inner circumferential surface of the main body,
wherein when the main body is unfolded to become a semicircle, a user can lay in the main body to get thermotherapy, and
wherein when the main body is folded and a sitting mat is mounted on the upper surface of the main body, the user can sit on the sitting mat to get thermotherapy.

2. The combined warmer and seat fumigator according to claim 1, further comprising:
fumigation modules respectively mounted on the inner circumferential surfaces of the first partitioned body and the second partitioned body.

3. The combined warmer and seat fumigator according to claim 2, wherein each of the fumigation modules comprises: a heating plate mounted in the first partitioned body and the second partitioned body, a container getting in contact with the heating plate accommodating herb medicines therein and having a discharge hole for discharging fumigation of the herb medicines into an internal space of the folded main body, and a cover for opening and closing an opening part of the container.

4. The combined warmer and seat fumigator according to claim 3, wherein the container or the cover is made of bio ceramic.

5. The combined warmer and seat fumigator according to claim 3, wherein the bioceramic is manufactured through the steps of:
(S1) crushing perlite and heating it at 850° C. to 950° C. in order to make foamed silica stone;
(S2) mixing and molding 20 to 40 parts by weight of inorganic binder based on 100 parts by weight of foamed silica stone; and
(S3) sintering the molded body through the step (S2) at 900° C. to 1,200° C.

6. The combined warmer and seat fumigator according to claim 5, wherein the inorganic binder of the step (S2) consists of 100 parts by weight of clay, 30 to 50 parts by weight of hollow body, 10 to 30 parts by weight of attapulgite, 5 to 15 parts by weight of foaming agent, and 50 to 70 parts by weight of water, and
wherein in the step (S3), the molded body is sintered to be foamed.

7. The combined warmer and seat fumigator according to claim 6, wherein the mixture of the step (S2) further includes 10 to 30 parts by weight of inorganic filler, which is at least one selected from diatomite, bentonite, zeolite, and alumina based on 100 parts by weight of the foamed silica stone.

8. The combined warmer and seat fumigator according to claim 7, wherein the hollow body is hollow silica powder having silica fume coated on the surface thereof, and
wherein the foaming agent is silicon carbide or calcium carbonate.
